(19) United States
(12) Patent Application Publication
(10) Pub. No.: US 2025/0224388 A1
(43) Pub. Date: Jul. 10, 2025

(54) ENVIRONMENTAL MONITORING SENSOR SYSTEM

(71) Applicant: Nooku Limited, Glasgow Strathclyde (GB)

(72) Inventors: Daniel Kane, Glasgow Strathclyde (GB); Gregor Aikman, Glasgow Strathclyde (GB); Craig Lynn, Glasgow Strathclyde (GB); Jonny Campbell, Glasgow Strathclyde (GB); Eilidh Price, Glasgow Strathclyde (GB); Stefan Raue, Glasgow Strathclyde (GB); Chamila Abeyratna, Glasgow Strathclyde (GB); Craig Easdale, Glasgow Strathclyde (GB)

(21) Appl. No.: 18/853,239

(22) PCT Filed: Apr. 4, 2023
(86) PCT No.: PCT/GB2023/050898

§ 371 Date: Oct. 1, 2024

(30) Foreign Application Priority Data
Apr. 4, 2022 (GB) 2204925.8

(51) Int. Cl. G01N 33/00; F16B 1/00; G08C 17/00

(52) U.S. Cl. CPC G01N 33/0075; G01N 33/0065; G08C 17/00; F16B 1/00; F16B 2200/83

(57) ABSTRACT

An environmental sensor system for measuring indoor air quality provides one or more modular base units (100) which are selectively coupled with one more main units (400) and which preferably also has a detachable cap (900) which can be used to change the mode of a user interface. to drive engagement and learning outcomes to drive better indoor air quality.

The present disclosure relates to an environmental monitoring sensor system, and in particular to an indoor air quality monitoring system; and to associated methods of monitoring environmental parameters and indoor air quality.

### BACKGROUND

With adults spending up to 90% of their time indoors, children are also spending much more time indoors. Children now spend half of the time outdoors that their parents did, only 68 minutes per day on average.

Children are also particularly vulnerable to the effects of poor indoor air quality as their lungs are still developing. Their airways are smaller, so inflammation caused by pollution can cause their airways to narrow more easily than is the case in adults. Currently one in 11 children and one in 12 adults are receiving treatment for asthma in the UK. This, coupled with an increasing amount of time being spent indoors due to COVID-19, means that indoor air quality has never been more of a concern for families and those in other indoor environments such as workplaces.

There are various air quality monitors available, but they tend to focus on sensing single pollutants and provide numeric information to the user which can be hard to interpret and draw conclusions from. The information provided by the sensors is not clear and not engaging so it risks people not being able to understand when they need to take action and doesn't motivate them to take action to improve air quality in the home or other indoor environment.

Furthermore, existing solutions are not tailored to different scenarios or different homes so there is a risk that they can generate false positives which tends to demotivate users over time.

While various large scale aggregate monitoring schemes are in place to monitor the quality of outdoor air quality, no similar intelligence exists for indoor air quality, so there is a lack of information for policy makers or other organisations such as governments, local councils, or large estate owners to understand data and trends and to inform policy and infrastructure investment.

Therefore, there is a need for improved environmental monitoring systems to facilitate and promote good health and practice in domestic and other indoor environments.

### SUMMARY

According to a first aspect of the disclosure there is provided an environmental sensor system comprising a plurality of sensor assemblies which include one or more base units and one or more main units, wherein the main unit is selectively engageable by hand with the base unit.

Preferably, the base unit comprises a first set of environmental sensors, and the main unit comprises a second set of environmental sensors.

Preferably, the main unit comprises a display screen and user interface.

Preferably, the system comprises actuation means for transforming the user interface of the main unit between a first mode and a second mode.

Each mode preferably provides a different set of menu display options and a different graphical look and feel for presentation to a user.

Preferably, the first mode comprises detailed information for display which is targeted at an adult or sophisticated user of the system, while the second mode displays a subset of the information shown in the first mode.

The second mode may comprise different graphical elements and different presentations designed to target different users, who may have different needs or preferences, or have different levels of permission to access data. One particular use case is to provide a more simplistic or engaging way of presenting information to a child and may include cartoon elements and modified language explaining the meaning of key terms. The second display mode may also provide a gamified interface.

Preferably, the actuation means comprises means for receiving a signal wirelessly from an associated computing device or web service.

Alternatively, the actuation means comprises a physical or capacitive switch provided at the body of the main unit.

Alternatively, the actuation means comprises a selectively attachable physical actuator member.

Preferably, the selectively attachable physical actuator member comprises a cap member which is selectively attachable to an upper portion of the main unit.

Preferably, the main unit and the selectively attachable physical actuator member are each provided with cooperating magnetic members for urging the members together and to ensure correct alignment when the members are placed together.

Preferably, the main unit and the selectively attachable physical actuator member are provided with a proximity air gap radio communication means, most preferably with a cooperating near field communication (NFC) tag and reader.

Preferably, the selectively attachable physical actuator member comprises a tag provided with a unique ID, and the user interface mode is selected based on the ID.

Preferably, the main unit and the base unit are each provided with cooperating magnetic members for urging the members together and to ensure correct alignment when the members are placed together.

Preferably, the main unit and the base unit are each provided with cooperating electrical actuators which enable communication of power and data between them.

Preferably, the base unit and/or the main unit are configured to communicate with a remote data platform.

Preferably, a base unit is provided with a location identifier and the base unit and main unit are configured to share this location identifier when they are connected.

Preferably, the location ID is stored at a remote platform.

Preferably, the base unit and main unit are configured to exchange network credentials upon connection, such that the main unit may automatically join a network when coupled with a base unit.

Preferably, the system is coupled with other devices which can affect air quality, and can generate control signals for these devices based on the air quality readings and/or recommended actions.

According to a second aspect of the disclosure, there is provided a method of sensing environmental parameters, comprising providing a plurality of sensor assemblies which comprise one or more base units and one or more main units, wherein the main unit can be selectively engageable with the base unit by hand.

The method of the disclosure may also comprise providing or using the system of the first aspect, or carrying out various other steps and processes as are described herein.

### BRIEF DESCRIPTION OF THE DRAWINGS

The present disclosure will now be described by way of example only, with reference to the accompanying drawings in which:

FIG. 1 shows a base unit of a system according to an embodiment of the disclosure;

FIG. 2 shows a further view of the base unit of FIG. 1;

FIG. 3 shows an exploded view of the base unit of FIG. 1;

FIG. 4 shows a main display and sensor unit of a system according to an embodiment of the disclosure;

FIG. 5 shows views of the main unit of FIG. 4 coupled with the base unit of FIG. 1;

FIG. 6 shows further views of the main unit and base unit combination of FIG. 5;

FIG. 7 shows an exploded view of the main unit of FIG. 4;

FIG. 8 shows an exploded view of selected components of the base unit of FIG. 1 and the main unit of FIG. 4, showing aspects of how the units cooperate for being coupled together;

FIG. 9 shows an actuator mechanism which can be selectively attached to the main unit of FIG. 4;

FIG. 10 shows views of the main unit of FIG. 4 coupled with the actuator mechanism of FIG. 9, and of the coupling together of all of the base unit of FIG. 2, main unit of FIG. 4 and actuator mechanism of FIG. 9;

FIG. 11 shows further views of the combination of base unit, main unit and actuator mechanism shown in FIG. 10;

FIG. 12 shows an exploded view of the actuator mechanism of FIG. 9;

FIG. 13 shows various exemplary display modes of the main unit of FIG. 4;

FIG. 14 shows further details of the base unit of FIG. 1;

FIG. 15 shows further details of the main unit of FIG. 4;

FIG. 16 shows an overview of a system architecture according to the disclosure;

FIG. 17 shows how a main unit can be commissioned;

FIG. 18 shows how a main unit may be tracked across different base units; and

FIG. 19 shows data sharing between the base unit and the main unit 400.

### DETAILED DESCRIPTION

According to the disclosure, an environmental sensor system comprises one or more base units which comprise a set of environmental sensors.

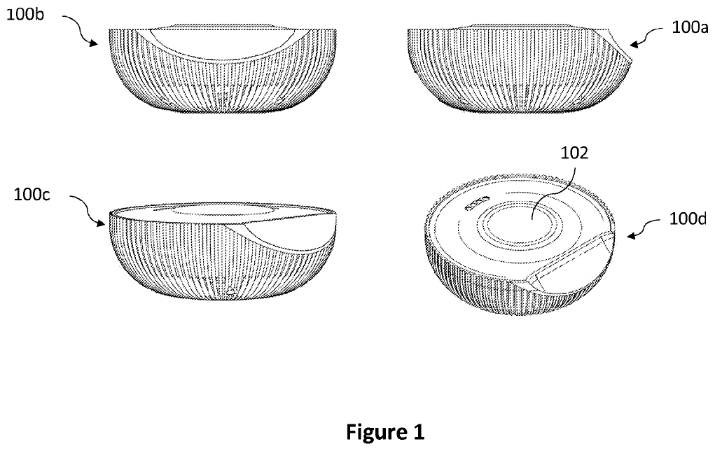

FIG. 1 shows an embodiment of a base unit 100 according to the disclosure, showing an isometric view 100d together with a left view 100a, front view 100b and a further perspective view 100c.

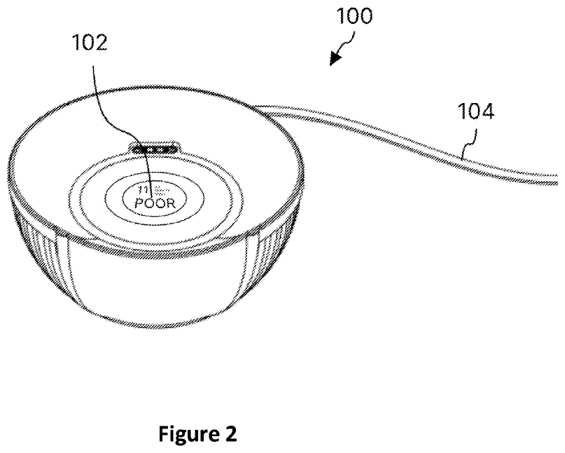

The base unit 100 comprises a set of base sensors. In one embodiment the set of base sensors may comprise one or more of: a volatile organic compound (VOC) sensor, sensors for detecting one or more types of nitrogen oxides (NOx) such as NO or NO₂, a temperature sensor, a relative humidity sensor, and an ambient light sensor. It will be appreciated that the specific set of sensors that is chosen to be incorporated in the base unit 100 may be different according to different specific implementations. The base unit 100 may also include a basic display functionality—see display 102. The base 100 may act as a stand-alone entry level sensor, and in one embodiment an environmental monitoring system is enabled by providing simply one or more of these base sensors. The display 102 may comprise a simple screen showing basic information. As shown in FIG. 2, this may provide some simple numeric information together with a general status report on air quality.

The base unit 100 may be enabled with wireless communication such as Wi-Fi, Bluetooth, or other wireless standards to connect with a mobile device or other network components, either via a central hub or via a mesh network.

The base unit is designed to be a relatively low cost unit which provides an entry level for new users and allows for future expandability. A user can benefit from some simple air quality monitoring by use of the base unit alone if they choose to do so.

The base may preferably be mains powered 104 as shown in FIG. 2. In an alternative embodiment it could be battery powered, but mains is preferred so that the base can reliably be powered for the long term.

As will be discussed in more detail below, the base unit is provided with a series of vents which permit airflow around the components.

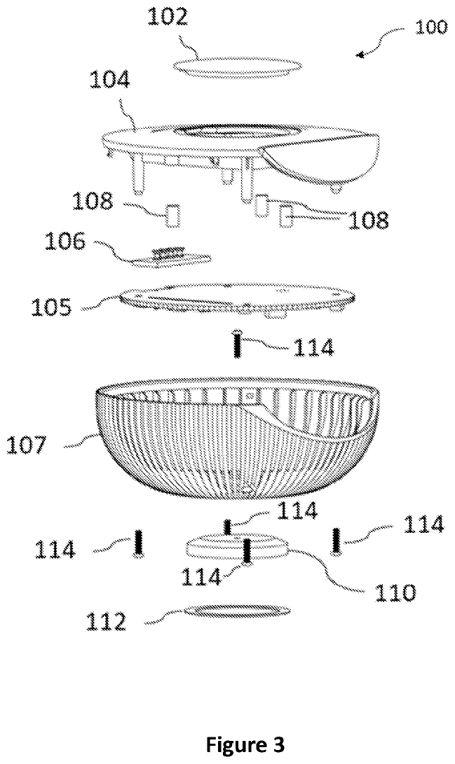

FIG. 3 shows an exploded drawing of the base 100 showing the various component parts.

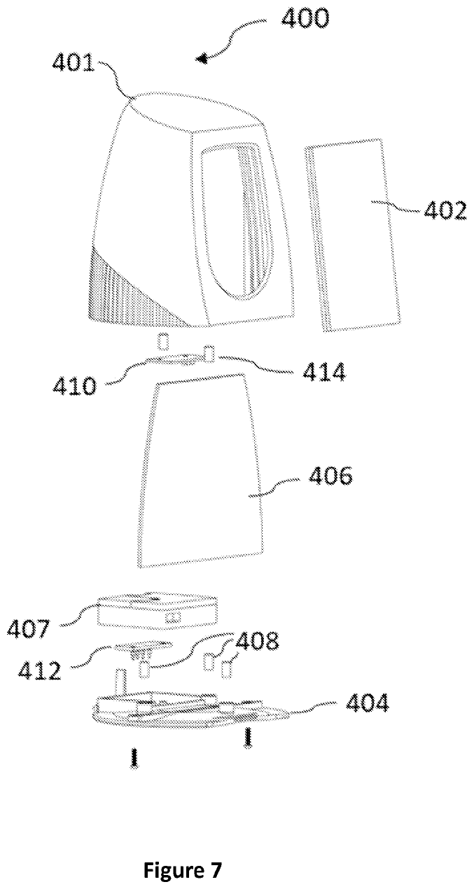

Screen member 102 is received within a lid member 104 which is mounted on a sensor/light emitting diode printed circuit board (PCB) 105. A connector PCB 106 includes contacts for an electrical connector, which in this embodiment is a pogo pin connector; here a set of targets which are provided for engagement with corresponding pin elements of a connector 413 of the main body 400 (see FIG. 7). These components are nested in the base enclosure 107 and a plurality of magnetic members 108 are provided for mating or cooperating with a corresponding set of magnetic members on the main body, as described elsewhere. A counterweight 110 is provided to provide mechanical stability, suitably being fabricated from steel or similar, and a rubber or similar high friction foot member 112 provides a high friction contact with a surface that the base unit 100 rests upon. The components are held together with fasteners 114, here in the form of threaded screws.

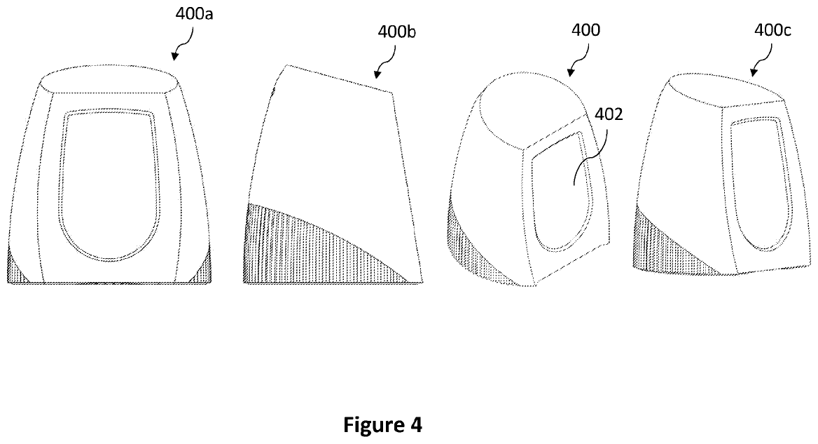

According to preferred embodiments, the system may also provide a main body unit 400 as illustrated in FIG. 4. This shows an isometric view of the main unit 400 alongside a front view 400a, a left view 400b and a further perspective view 400c.

The main body 400 comprises a second set of sensors which may provide additional functionality as compared with the set of sensors in the base unit 100. The second set of sensors may also include sensors which perform the same function as one or more of the sensors in the base unit 100, so that certain key parameters can always be monitored irrespective of whether the main unit is provided or not as part of the overall system, and can be monitored at all times in all locations where sensor assemblies are present. As a non-limiting example embodiment, the second set of sensors may comprise one or more of: particular matter sensors, a carbon dioxide sensor, a temperature sensor, a relative humidity sensor, a motion sensor (such as an accelerometer), and an orientation sensor. The particulate matter sensors may comprise one or more of: a PM 1.0 sensor, a PM 2.5 sensor, a PM 4.0 sensor and a PM 10 sensor, or sensors designed to target other particulate matter concentrations. The main unit 400 also comprises a screen member 402 which may optionally be a touch screen.

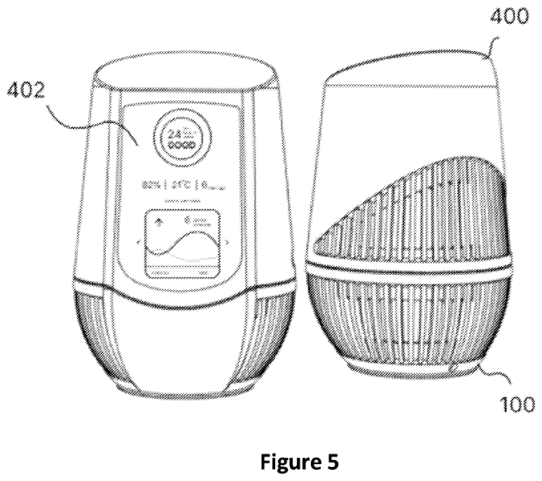

FIG. 5 shows an example information display on the touch screen 402. FIG. 5 shows the main body 400 coupled with a base unit 100 of the type shown in FIGS. 1 and 2.

The main body 400 is designed to fit neatly to the base 100 and can be easily moved from base to base. It can be selectively attached and detached by hand, without requiring any tools.

A typical system implementation in a domestic environment may include a plurality of base units 100 and a single main body 400. However, it will be appreciated that in alternative embodiments a plurality of main units 400 may be provided, including providing one main body 400 for every base unit 100. However, having a single main unit 400 that can be moved flexibly between different base units 100 provides a cost-effective way for a consumer to benefit from the system.

The main unit 400 can be moved to different locations during the day based upon where people will be present in the dwelling or internal environment; for example being moved from a living room during the day to a bedroom at night so that more data can be collected.

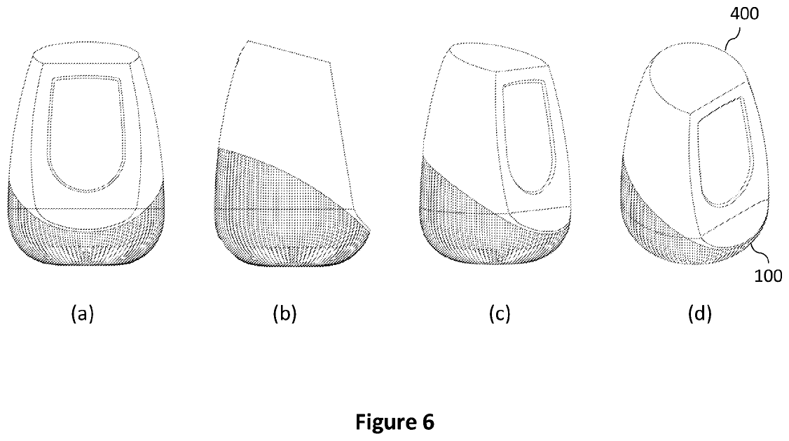

FIG. 6 shows a further illustration of base 100 and main body 400 coupled together, with the isometric view (d) being accompanied by a front view (a), a left view (b) and a further perspective view (c).

The main body 400 may be selectively coupled with the base unit 100. The modules need to be connected in a mechanically secure way that is visually unobtrusive. Furthermore, the main body 400 and the base 100 also need to transfer data between each other.

Therefore, the main body 400 and base unit 100 may be selectively coupled by use of a connection means which may include an electrical connection means together with one or more of a magnetic connection means and physical connection means.

The electrical connection means may comprise an electrical connector, which may preferably comprise a spring mounted pogo pin mechanism whereby one of the two parts to be connected is provided with a set of target connection surfaces and the other is provided with a set of pin members-generally herein, if a pogo pin arrangement is shown in one target and pin configuration, the targets and pins may be reversed if desired.

The magnetic connection means may comprise one or more cooperating pairs of magnetic members, the members of the or each pair being provided at specific matching locations in each of the devices to be connected.

The magnetic members may preferably be provided within the housing of each device, positioned just below their respective surfaces to provide a strong magnetic coupling but being invisible to the everyday user.

The cooperating magnetic members are used to connect the devices in an orientation which self-locates, guiding the user to the correct arrangement.

FIG. 7 shows an expanded view of the main body 400. The main enclosure 401 receives a screen member 402 and is connected to a lower lid member 404.

The housing 401 encloses a main body sensor printed circuit board (PCB) 406 and a particulate matter (PM) sensor 407 which is preferably isolated from the main PCB 406 for airflow reasons. A further NFC connector printed circuit board 410 is provided which is for electrical communication with the cap member 900 in cases where this is provided. A first set of magnetic members 408 are provided for ensuring mechanical connection with the base 100 as described elsewhere and an electrical connector PCB 412 is provided for electrical connection with the base 100.

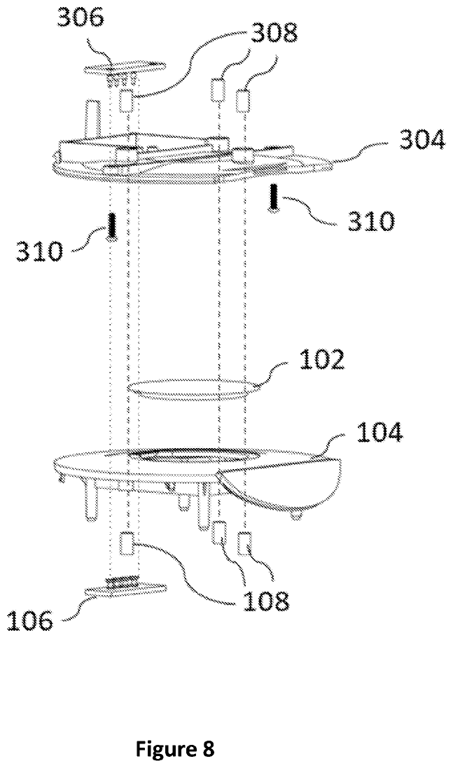

FIG. 8 shows the cooperation between a base portion 304 of the main body 300 and a lid member 104 of the base unit 100. As can be seen here, a plurality of magnets 308 in the main unit 300 are positioned to locate with corresponding magnets 108 provided within the body of the base unit 100 and an electrical connector comprises a pogo pin mechanism comprising a set of pin members 306 provided in the main body 300 which mates with a set of target members 106 provided in the base lid. Screw members 310 also are used as part of the main body to hold it together.

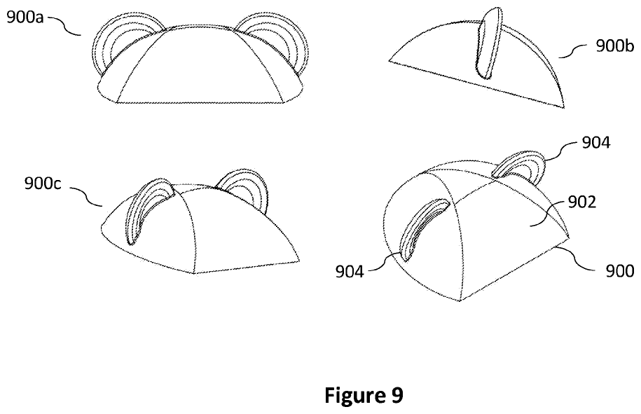

In a preferred embodiment, the system may also comprise a physical actuator member 900 as shown in FIG. 9. Here, an exemplary physical actuator member is provided in the form of a cap, as shown in the isometric view 900, which is shown together with a front view 900a, a left view 900b and a further perspective views 900c.

The cap member 900 is designed to selectively engage with the main body unit 300 and is designed so that the appearance of the device can transform to have a more engaging appearance that will appeal to children, or generally to provide a more aesthetically pleasing effect. In this example the cap member 900 has a main body 902 provided with a pair of ear portions 904.

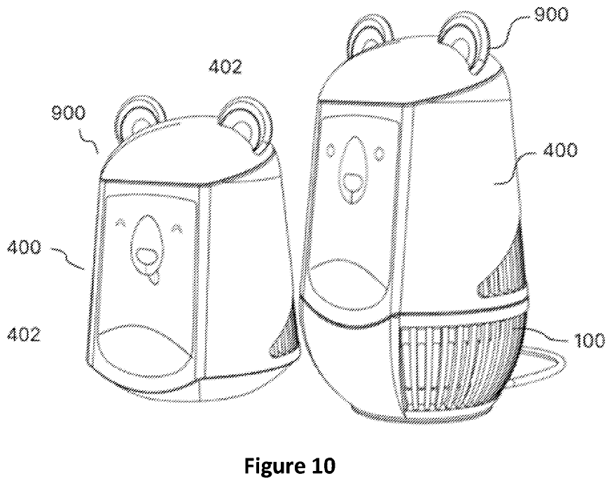

The selective attachment of the cap member 900 with the base 400 is used as a trigger to transform the operational mode of a user interface that is provided via the display 402 of the main body 400. This may include transforming the user interface to a special child mode and as can be seen in FIG. 10, this special mode may include graphical elements that complete the look of a special character that matches the physical design of the cap member 900.

The system may be provided with a range of different cap members 900 that may have different personalities or different designs to enable to cater for different tastes or different franchising opportunities for engagement of different audiences. The cap member 900 connects magnetically with the main body 400, as described in more detail elsewhere herein.

The child mode engages children at a level that they can understand and can educate and entertain them. The main body 400 can also be removed from the base to allow more play/exploration opportunities.

When the cap 900 is attached, it should signify to both the main body 400 and the base unit 100 that it is present. Sub-surface magnets are provided in a cooperating fashion at the main body 400 and cap 900 to ensure a natural registration of the cap in the correct orientation.

The cap may use an RFID tag or NFC connection to alert the main body 400 that it has been attached.

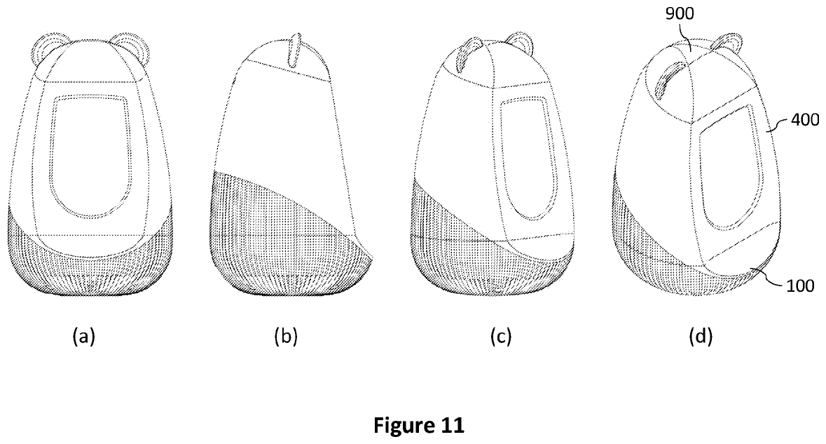

FIG. 11 shows further details of how the base unit 100, main unit 400 and cap member 900 may be connected together, showing an isometric view (d), along with front view (a), left view (b) and further perspective view (c).

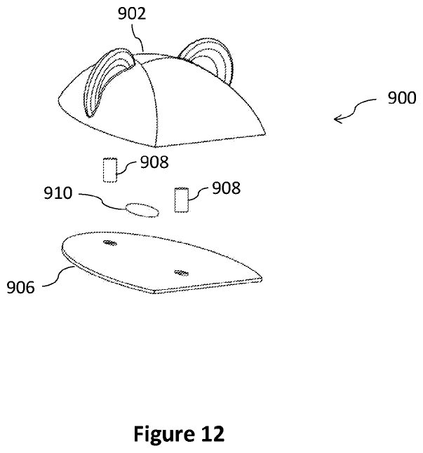

FIG. 12 illustrates an exploded drawing of the cap member 900. Here, the cap enclosure 902 is coupled with a cap lower lid member 906 and comprises a plurality of magnet members 908 which are designed for registration with corresponding magnet members 414 of the main body 400. An NFC connector PCB 910 is also provided for communication with the main body 400.

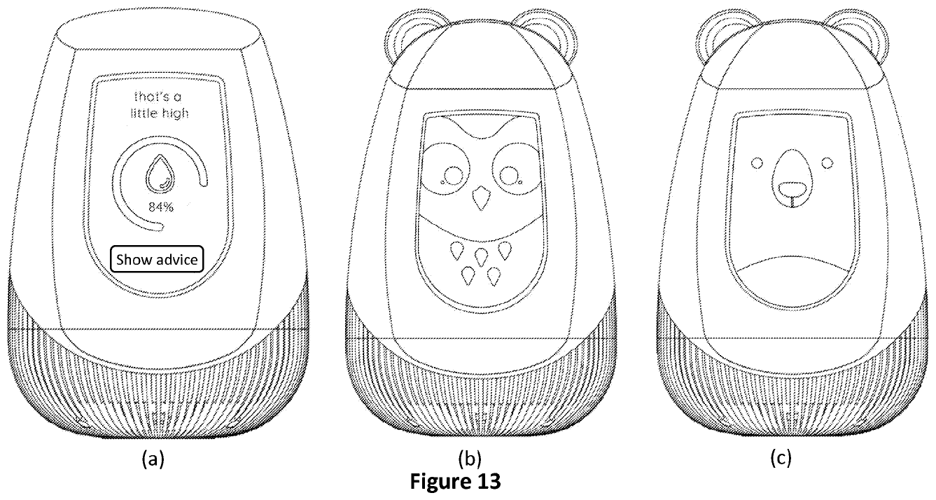

FIG. 13 shows examples of how different operational user interface modes may be presented depending on the selective attachment of the cap member 900.

In FIG. 13 (a) the cap member is not attached and the user interface is in a fully featured mode suitable for use by adults or other sophisticated users. It provides data but still in a clear way with suggested actionable improvements. But when the cap 900 is attached, the user interface can change to a child character which can interact with the child to educate and entertain. Figures (b) and (c) show options for different characters that may be provided. Different characters can be provided based on the age or preferences of the user.

In alternative embodiments, different user interface modes can be triggered without the need for a physical actuator. For example, the mode could be switched by sending a command from a remote control or companion mobile application on a smartphone or PC. Furthermore, the physical actuator could comprise a physical actuator such as a push button, rocker switch or touch sensitive element provided at the surface of either of the base unit or the main unit. Where a selectively attachable physical actuator member is provided, it may be selectively attached to other portions of the main unit such as its sides, or to the base unit, i.e. it is not essential that the selectively attachable physical actuator member is attachable to the upper surface of the main unit, or that it has the form of a cap member.

In a system like this, various components generate substantial amounts of heat. This heat can interfere with temperature and humidity sensors in the modules. Therefore, various features have been provided to ensure flow of heat to minimise or eradicate this problem.

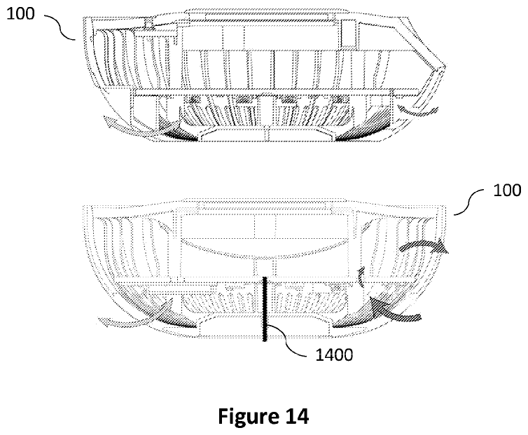

FIG. 14 shows solutions that can be provided as part of the base. Here a small pocket is created between the base enclosure and the printed circuit board using ribs that protrude through the printed circuit board. This isolates components within the pocket from the air in the rest of the base 100 and also creates a break in the PCB to prevent conduction through the board. All components within the pocket are primarily exposed to ambient air only. These components may be including an ambient light sensor.

An additional vent is placed in the circuit board to isolate the other sensor components from the main heat generating components and a rib member 1400 may be provided to separate the base enclosure into two sections. The vent also serves as a chimney to draw warm air from beneath the circuit board past the less sensitive components to aid detection of air quality.

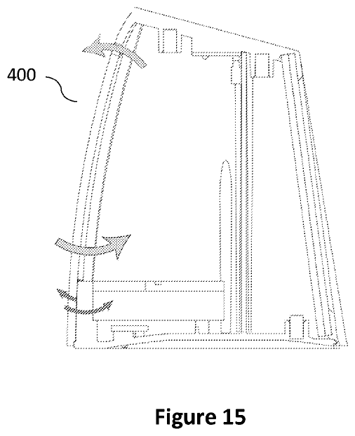

FIG. 15 shows how a chimney effect can also be created within the main body 400 to draw air past components on the printed circuit board. The in and out vents on the particulate matter sensor may also be isolated with a rib member to help avoid cross contamination.

As mentioned above, the base unit may comprise a basic set of sensors. The main unit may comprise a comprehensive sensor suite for measuring a range of different pollutants and environmental factors. These may include sensors for measuring temperature, humidity, volatile organic compounds, equivalent carbon dioxide, carbon monoxide, nitrogen dioxide, and particulate matter, including for example PM 1, PM 2.5 and PM 10 sensors.

Sensors in the base unit and in the main unit may cooperate when they are provided together to provide increased intelligence.

The system may also provide smaller, low-cost peripheral sensors in order to build a more complete air quality map of the entire home.

The modular nature of this system provides an affordable means to buy into the product ecosystem and can be added to and upgraded cost-effectively when new features and sensor technologies become available.

The system including the sensors can also communicate wirelessly or otherwise to transmit sensor data for a user to view and monitor the information. The system may include an application running on a mobile device or as a web application that can provide insight and be a companion to the modules of the system.

At a basic level, the system may provide an alert when certain thresholds of the measured parameters are reached, crossed, or exceeded. However, in a preferred embodiment the system can learn the individual needs of each household in which it is provided and it can tailor feedback and advice appropriately. These recommendations may range from immediate quick fixes to more insightful, longer term occupant actions driven by analysis of trends in the data.

This may be achieved during setup when a range of occupant dwelling and environmental data is gathered allowing the system to adjust the nature and the intensity of actionable advice given. The data that is gathered may include information about the furniture, windows, heating and ventilation systems in a building, and information about health conditions of family members, such as asthma or other breathing conditions. These data can be used to tailor the recommendations and tailor the triggering of alerts depending on specific requirements.

The data gathered in this set-up survey allows specific air quality needs of a user or group of users (such as a family) to be understood. From this, the system's alerts and advice can be automatically adjusted to provide a tailored solution for everyone. For example, someone with asthma may be more sensitive to particulate matter (PM), so if a user with this condition is in the household more weight can be given to PM readings, with encouragement to rectify any problems delivered at lower thresholds; or if there are young children in the home then the system can ensure their bedroom does not reach conditions which could be potentially harmful to their respiratory development. This is an improvement over existing systems, which provide a blanket one-size-fits-all approach to air quality feedback. This is often not appropriate for everyone, especially when allergies or other respiratory conditions are involved. The system allows monitoring to be tailored, in cognisance that every person is different, with their own unique combination of problems and sensitivities.

The system may use artificial intelligence and machine learning techniques to learn trends and spot when an event occurs that is different from trends. This means that the system can become smarter over time and reduce false positives as it begins to recognise relationships between changes in air quality, pollution events and user behaviour.

Furthermore, the system can also be linked to other devices which can affect air quality, and can generate control signals for these devices to automatically intervene and take action to improve the indoor air quality. For example, the system could automatically activate a purifier, a dehumidifier, an HVAC system, or open or close windows based on the air quality readings, or to physically implement the recommended actions that are suggested by the system. These actions could be taken automatically, or could be pre-approved by a user, for example by sending a notification to them on a smartphone app or similar.

Data from the system can be collated and aggregated and made available to other users such as government bodies, local councils and academic institutions. The data can be collected with clear consent of the users and is anonymized. This means that policymakers can obtain data about indoor air quality that will inform policy decisions, such as introducing or maintaining regulations around household ventilation, or traffic regulation. This may also be paired with occupant demographic and health data from the device setup survey, so it will create a valuable data set on a scale which is not achievable through existing approaches.

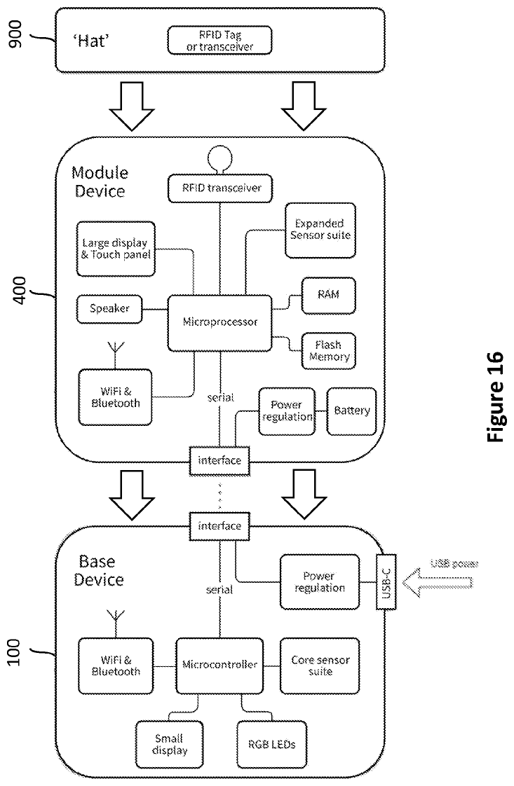

The system can also be understood in terms of the underlying system architecture, which is illustrated in FIG. 16, showing the base unit 100, main unit 400 (also known as a “module device”) and physical actuator member 900 (referred to here as a hat).

As can be seen here, the base unit 100 comprises a microcontroller arranged to communicate over a serial communications channel with an interface, and which communicates with a wireless communication module (Wi-Fi and Bluetooth), display unit and a set of indicator elements such as red-green-blue light emitting diodes for providing user feedback/status information. The base unit also comprises a power input (USB-C in this example) and power regulator. The microcontroller is also coupled with a first set of sensors, a core sensor suite, which are discussed elsewhere herein.

The main unit 400 can be selectively coupled with or decoupled from the base unit 100, and comprises an electrical interface for coupling with the respective interface of the base unit 100. The main unit 400 is provided with an internal power source such as a rechargeable battery, and a power regulator. The functions of the main unit 400 are controlled by a core microprocessor and memory comprised of non-volatile memory that is suitably FLASH, and volatile random access memory (RAM). The microprocessor is communicatively coupled with the interface by a communications bus, suitably a serial interface, and also with other components including a wireless communication module (Wi-Fi and Bluetooth), large display unit and touch panel, speaker, RFID transceiver/NFC reader and an expanded sensor suite, as discussed elsewhere herein.

The physical actuator member 900 may be provided with a tag, suitably an RFID tag or transceiver, which may preferably be provided with a unique ID. The behaviour of the system may depend on the unique ID, for example, by changing the display mode or user interface appearance.

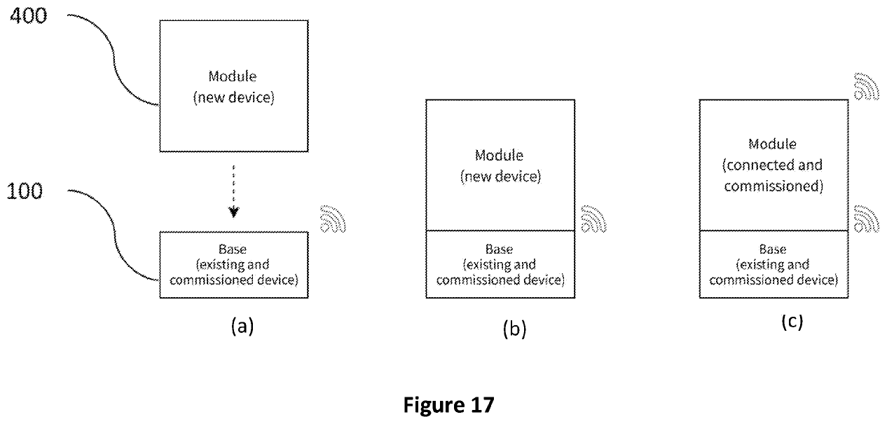

When a main unit 400 is to be set up with a new system, it must be commissioned so that it can function together with other devices in the system. Commissioning new internet of things (IoT) devices is often an involved, manual process for the user. FIG. 17 illustrates how the main unit 400 can be commissioned, preferably in an automated fashion.

Here, a newly purchased main unit 400 can simply be dropped onto an existing base unit 100 and it will automatically commission itself. Using the bidirectional communication interface with the base 100, the main unit 400 can request current and valid Wi-Fi or other network credentials, as well as other relevant pieces of information (User/System ID) allowing it to commission itself for immediate use.

Part (a) of FIG. 17 shows base 100 connected to network and commissioned within user's home. Then in part (b), a main unit 400 is mounted on the base 100. The base unit 100 then communicates active network connection credentials and user ID to the main unit 400, with then (in part (c)) connects to the network with new credentials and commissions itself with the user ID provided.

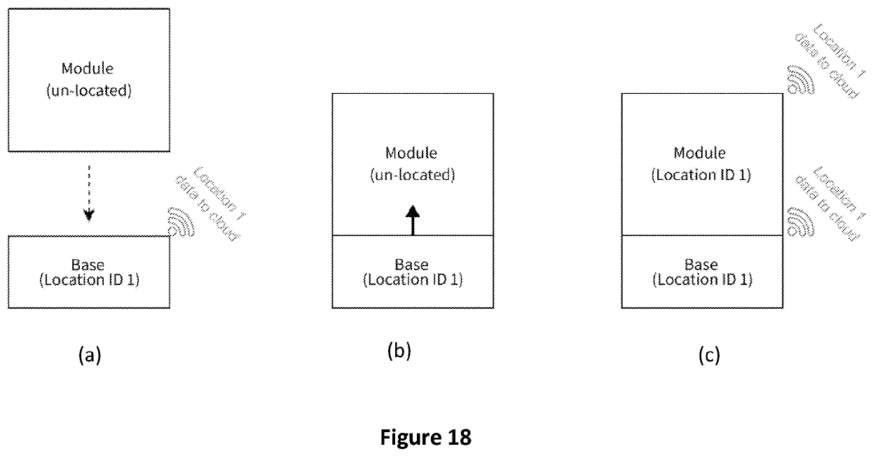

The main unit 400 may also be moved around to be fitted to different base units 100. The system can track this, as shown in FIG. 18. It is desirable for the base unit 100 to know its location, so that location-accurate information can be provided as the main unit moves from base to base. To deal with this, a bidirectional communication interface between base unit 100 and main unit 400 is provided. When the main unit 400 is placed atop a new base unit 100, that base unit 100 communicates its unique ID. This ID is linked to a static location know by the system and recorded on a data platform (accessed by application or web interface), meaning that the module can use this new ID to communicate sensor readings and have them linked to its current location.

FIG. 18 shows in part (a) the base unit 100 transmitting location specific sensor data, preferably to a cloud platform provided as part of the system or for communication with the system, with the main unit 400 unmounted. In part (b), the main unit 400 is then mounted on the base unit 100 and the base unit 100 communicates its location ID to the main unit 400. The module then (at part (c)) adopts the location ID and begins transmitting location specific data.

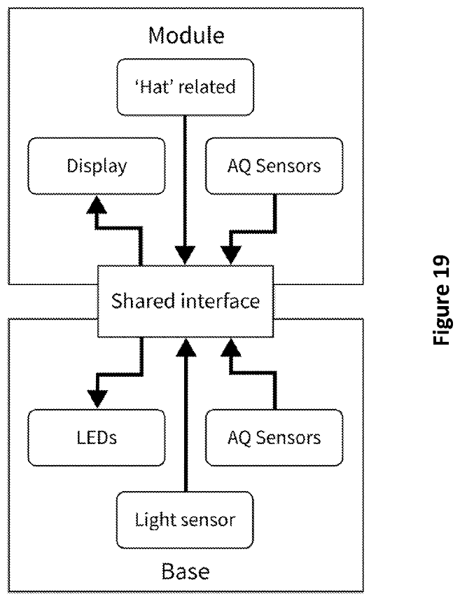

The system of the disclosure also makes it possible to share data in real time between the base unit 100 and the main unit 400, as shown in FIG. 19.

It is desirable for rapidly updating real time data to be displayed by the module, but it is typically saved to the cloud platform at lower intervals than required. User air-quality indicia (such as lights, here provided by LEDs), may vary to show the overall air quality (AQ). However, sensors to control the brightness of the LEDs and display are located only in the base. To deal with this, the communication interface provides a means for base 100 and main unit 400 to share real-time information at higher frequencies than are sent to the cloud. Air quality sensor data from the base 100 is sent to the main unit 400 to update its display. Air quality sensor data from the main unit 400 is sent to the base unit 100 to update the overall air quality index, translating for example to different base LED colour indications. The presence of a character ‘hat’ or other physical actuator member 900 can also be translated from the main unit 400 to the base 100 to alter base LED output. The base unit 100 may also send ambient light details to the main unit 400 to adjust its display brightness or change to ‘dark mode’ in the absence of light.

As mentioned above, the physical actuator member 900 may be provided with a unique user ID. This can enable the saving of progress of a “character journey” which can be customised for different people, such as different members of a household. Each unique ID can be logged to track progress and to save this progress to a remote platform. In this way, disconnection and later reattachment of the hat will allow the user to resume whatever game or ‘journey’ the character is guiding them on. Multiple users in a household with their own hat will be able to have their own independent progress. This could work within one home, as well as on systems in others' homes.

Various improvements and modifications could be made to the above without departing from the scope of the present invention.

1. An environmental sensor system comprising a plurality of sensor assemblies which include one or more base units and one or more main units, wherein the main unit is selectively engageable by hand with the base unit.

2. The system of claim 1, wherein the base unit comprises a first set of environmental sensors, and the main unit comprises a second set of environmental sensors.

3. The system of claim 1, wherein the main unit comprises a display screen and user interface.

4. The system of claim 1, comprising actuation means for transforming the user interface of the main unit between a first mode and a second mode.

5. The system of claim 4, wherein the first mode comprises detailed information for display which is targeted at an adult or sophisticated user of the system, while the second mode displays a subset of the information shown in the first mode.

6. The system of claim 4, wherein the actuation means comprises means for receiving a signal wirelessly from an associated computing device or web service.

7. The system of claim 4, wherein the actuation means comprises a physical or capacitive switch provided at the body of the main unit.

8. The system of claim 4, wherein the actuation means comprises a selectively attachable physical actuator member.

9. The system of claim 8, wherein the selectively attachable physical actuator member comprises a cap member which is selectively attachable to an upper portion of the main unit.

10. The system of claim 8, wherein the main unit and the selectively attachable physical actuator member are each provided with cooperating magnetic members for urging the members together and to ensure correct alignment when the members are placed together.

11. The system of claim 8, wherein the main unit and the selectively attachable physical actuator member are provided with a proximity air gap radio communication means, most preferably with a cooperating near field communication (NFC) tag and reader.

12. The system of claim 11, wherein the selectively attachable physical actuator member comprises a tag provided with a unique ID, and the user interface mode is selected based on the ID.

13. The system of claim 1, wherein the main unit and the base unit are each provided with cooperating magnetic members for urging the members together and to ensure correct alignment when the members are placed together.

14. The system of claim 1, wherein the main unit and the base unit are each provided with cooperating electrical actuators which enable communication of power and data between them.

15. The system of claim 1, wherein the base unit and/or the main unit are configured to communicate with a remote data platform.

16. The system of claim 1, wherein a base unit is provided with a location identifier and the base unit and main unit are configured to share this location identifier when they are connected.

17. The system of claim 16, wherein the location ID is stored at a remote platform.

18. The system of claim 1, wherein the base unit and main unit are configured to exchange network credentials upon connection, such that the main unit may automatically join a network when coupled with a base unit.

19. The system of claim 1, being coupled with other devices which can affect air quality, and can generate control signals for these devices based on the air quality readings and/or recommended actions.

20. A method of sensing environmental parameters, comprising providing a plurality of sensor assemblies which comprise one or more base units and one or more main units, wherein the main unit can be selectively engageable with the base unit by hand.
